(19) United States
(12) Patent Application Publication
(10) Pub. No.: US 2023/0107113 A1
(43) Pub. Date: Apr. 6, 2023

(54) NASAL DEVICE AND ASSOCIATED METHOD OF MEASURING AN ASPECT OF GAS FLOWING THROUGH A USER'S NOSE

(71) Applicant: Mission Support and Test Services, LLC, Las Vegas, NV (US)

(72) Inventors: Scott B. Sonnon, North Las Vegas, NV (US); Erik A. Stassinos, North Las Vegas, NV (US)

(21) Appl. No.: 17/958,821

(22) Filed: Oct. 3, 2022

Related U.S. Application Data
(60) Provisional application No. 63/251,988, filed on Oct. 4, 2021.

(51) Int. Cl. A61B 5/00; A61B 5/08; A61B 5/083; A61B 5/097

(52) U.S. Cl. CPC A61B 5/6819; A61B 5/0803; A61B 5/0833; A61B 5/0836; A61B 5/097

(57) ABSTRACT

A nasal device is provided to measure an aspect of gas flowing through a user's nose. The nasal device includes a body having a first interior side and a second, opposing interior side. The body forms a lumen between the first interior side and the second interior side through which the gas flows into and out of the user's nose. The nasal device further includes an emitter coupled to the first interior side and configured to emit emitted energy, a detector coupled to the second interior side and configured to receive detected energy, and a wireless transceiver coupled to the body and configured to transmit data corresponding to at least one of the emitted energy and the detected energy.

### FIELD OF THE INVENTION

The present invention is directed to a nasal device and an associated method of measuring an aspect of gas flowing through a user's nose using the nasal device.

### DESCRIPTION OF THE RELATED ART

Physiological trends are indicative of human health. Current methods of discovery of physiological trends revolve around the measurement and analysis of oxygen saturation of the blood and the pulse rate, such as via a pulse oximeter. Specifically, oxygen saturation gives information about the amount of oxygen carried in the blood. Pulse oximeters thus can estimate the amount of oxygen in the blood without having to draw a blood sample.

However, physiological trends based on oxygen saturation are inaccurate during high performance activities in the human body because, due to breathing-related changes, more oxygen may be bound to hemoglobin, resulting in a measured increase in the percentage oxygen saturation percentage. Thus, physiological trends typically associated with a prediction of low performance or illness (e.g., as indicated by lower oxygen saturation levels) may be confusing and inaccurate when the human body is engaged in high performance activity. For example, a higher oxygen saturation level is not an indication of better physiological performance, if more oxygen remains bound to blood hemoglobin and less available for oxygen delivery to performing tissues.

In contrast, carbon dioxide is a more accurate measure of physiological trends. In capnography, carbon dioxide exhaled from the human body is in a tightly regulated range, between 35 mm to 45 mm, measured by end-tidal carbon dioxide (ETCO2) levels. Carbon dioxide levels ranging above 45 mm ETCO2 are indicative of an excess of lactic acid (hypercapnia), and thus predictive of muscular fatigue, and carbon dioxide levels ranging below 35 mm ETCO2 (hypocapnia) is predictive of muscular weakness. Studies have shown changes in carbon dioxide levels may not only predict muscle fatigue and weakness, but also conditions involving the other organs and tissues. Although the medical field has been using large, fixed/wired apparatus capnography with nasal canula tubes to measure ETCO2 levels for patient diagnostics when in hospital beds, improved apparatus and methods are desired, as carbon dioxide may offer insights into early detection of trends toward specific illnesses and diseases. It is with respect to these and other considerations that the disclosure made herein in presented.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1A is an isometric view of a nasal device, in accordance with one non-limiting embodiment of the disclosed concept.

FIG. 1B shows the nasal device of FIG. 1A employed with a user.

FIG. 2 is an isometric view of another nasal device, in accordance with another non-limiting embodiment of the disclosed concept.

FIG. 3 is a simplified view of another nasal device, showing one or more internal components, in accordance with another non-limiting embodiment of the disclosed concept.

FIG. 4 is a simplified view of another nasal device, shown in an exemplary use condition, in accordance with another embodiment of the disclosed concept.

FIG. 5 illustrates an exemplary plot of carbon dioxide absorbance relative to wavelengths.

FIG. 6A shows a simplified view of the nasal device of FIG. 2, and shown without the presence of gas molecules.

FIG. 6B shows another simplified view of the nasal device of FIG. 2, and shown with respiration passing through a lumen of the nasal device.

FIG. 6C shows the nasal device of FIG. 1A, and shown with respiration passing through two lumens of the nasal device, in accordance with another non-limiting embodiment of the disclosed concept.

FIG. 6D shows an exemplary graph of carbon dioxide measured when respiration passes through the lumen of the nasal device of FIG. 2.

FIG. 6E shows an exemplary graphical display of the carbon dioxide measured when respiration passes through the lumen of the nasal device of FIG. 2, and shown during inspiration and expiration.

FIG. 6F shows an exemplary graphical display of the carbon dioxide measured when respiration passes through the lumen of the nasal device of FIG. 2 over a period of time.

### SUMMARY OF THE INVENTION

In accordance with one aspect of the disclosed concept, a nasal device is provided. The nasal device measures an aspect of gas flowing through a user's nose, the nasal device comprising: a body having a first interior side and a second, opposing interior side, the body forming a lumen between the first interior side and the second interior side through which the gas flows into and out of the user's nose; an emitter coupled to the first interior side and configured to emit emitted energy; a detector coupled to the second interior side and configured to receive detected energy; and a wireless transceiver coupled to the body and configured to transmit data corresponding to at least one of the emitted energy and the detected energy.

In accordance with another aspect of the disclosed concept, a method of measuring an aspect of gas flowing through a user's nose using a nasal device is provided. The method comprises the steps of: directing the gas through a lumen of the nasal device, the nasal device being disposed in the user's nose; emitting a first amount of energy with an emitter of the nasal device, the emitter being coupled to a first interior side of a body of the nasal device; detecting a second amount of energy received at a detector of the nasal device, the detector being coupled to a second interior side of the body of the nasal device, the second interior side being disposed opposite the first interior side; comparing the first amount to the second amount to calculate an absorbed energy amount; generating data based on the absorbed energy amount; and transmitting the data with a transceiver of the nasal device.

### DETAILED DESCRIPTION OF THE PREFERRED EMBODIMENTS

Disclosed is a capnography apparatus and method to measure one or more aspects of gas exhaled through the nose while having ambulatory freedom from tubes and wires. In other words, the apparatus of the disclosed concept, e.g., capnographic dioximeter nasal devices (“nasal devices”) 102, 202, 302, 402, are configured as self-contained subassemblies that are advantageously devoid of external wires and tubes. In one embodiment, a nasal device is inserted into a person's nostrils to measure one or more aspects of gas exhaled and/or inhaled by the wearer of the device. One or more sensors may be embedded in the nasal device to detect and measure exhaled end-tidal carbon dioxide, or any other gas (e.g., oxygen).

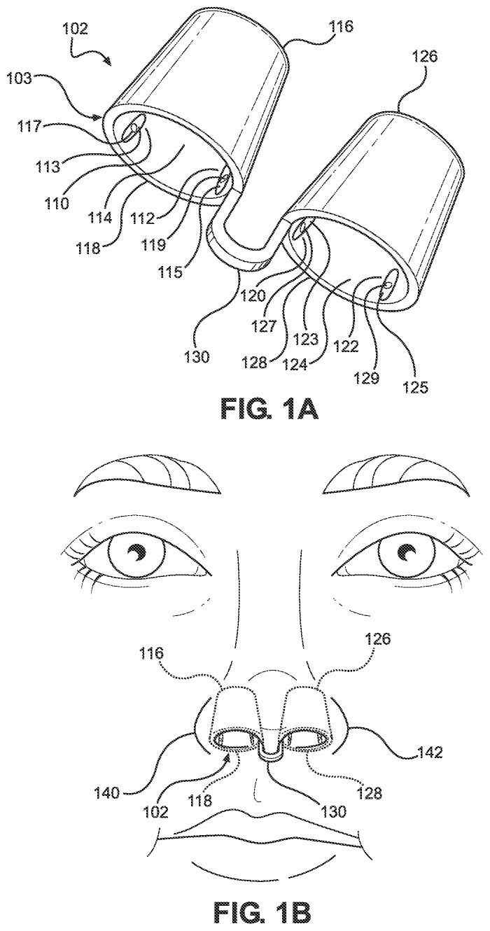

FIG. 1A illustrates an exemplary nasal device 102, and FIG. 1B shows the nasal device 102 inserted into a person's nostrils 140, 142. In one example embodiment, and as will be discussed in greater detail below, the nasal device 102 is configured to measure an aspect of gas flowing through a user's nose. The nasal device 102 includes a body 103 having a pair of opposing interior sides 110, 112, 120, 122. It will be appreciated that the body 103 forms a corresponding pair of lumens 114, 124 with the first lumen 114 being between the first and second interior sides 110, 112, and the second lumen 124 being between the third and fourth interior sides 120, 122.

Continuing to refer to FIG. 1A, the body 103 of the nasal device 102 may also include corresponding first and second openings 116, 118, 126, 128 at corresponding ends of the first and second lumens 114, 124. As such, gas (e.g., carbon dioxide and oxygen) may flow into and out of the user's nose through the first and second lumens 114, 124. The first openings 116, 126 may be smaller than the second openings 118, 128, e.g., may have a smaller diameter, in order to match the internal shape of the nasal passage of the nose. The nasal device 102 may be inserted into the person's nostrils 140, 142 such that the first openings 116, 126 reach the far end of the nostrils 140, 142, and the second openings 118, 128 may be located closer to the end of the nostrils 140, 142.

The body 103 may also include a bridge 130 connecting the first lumen 114 to the second lumen 124. Additionally, in one example embodiment the body 103 is a single unitary component made from one piece of material (e.g., without limitation, is an injection molded piece). It is contemplated that the nasal device 102 may assume different shapes and sizes such that any size or shape of device may be used that is suited for monitoring the incoming and outgoing gas from a person's respiration through a nasal passage.

In accordance with the disclosed concept, the nasal device 102 has internal components that allow aspects of respiration to be measured. In one example embodiment, and as shown in FIG. 1A, the body 103 of the nasal device 102 may have any number of internal compartments 113, 115, 123, 125 for holding electronic equipment (e.g., emitters 117, 127 and detectors 119, 129, shown in simplified form). Accordingly, the emitters 117, 127 may be coupled to the corresponding first interior sides 110, 120 and the detectors 119, 129 may be coupled to the corresponding second interior sides 112, 122. It will also be appreciated that a nasal device in accordance with the disclosed concept may present with a single lumen instead of the two lumens 114, 124 disclosed in connection with FIG. 1A.

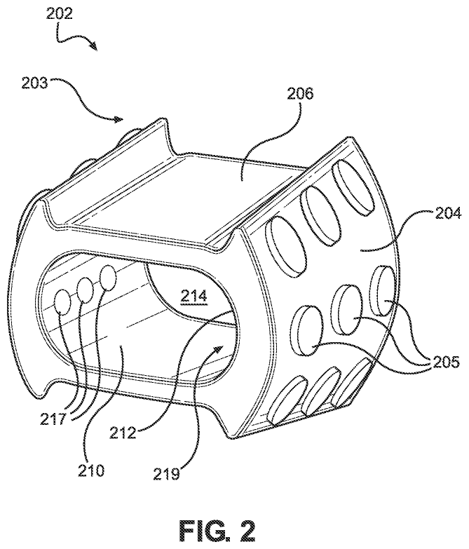

For example, FIG. 2 shows another nasal device 202. It will be appreciated that the nasal device 202 may likewise have internal compartments (not labeled) for housing electronic equipment in the same manner as the nasal device 102, described above. More specifically, as partially shown in FIG. 2, coupled to a first interior side 210 of a body 203 of the nasal device 202 (e.g., in an internal compartment) are one or more emitters 217, and coupled to a second interior side 212 of the body 203 (e.g., in an internal compartment) are one or more detectors (not shown, but located proximate reference numeral 219), each located on an opposite side of the lumen 214.

The one or more emitters 217 are configured to emit emitted energy (e.g., without limitation, light whether in the visible or non-visible spectrum, or other types of energy, such as LED emitters). The one or more detectors 219 are configured to receive detected energy (e.g., wavelength of light or any other type of energy emitted from the emitters 217 after passing across the gap between the emitters 217 and the detectors 219). Carbon dioxide molecules may block or absorb the light or any other type of energy emitted from the emitters 217, causing the detectors 219 to receive a smaller amount of light or any other type of energy than is emitted. By comparing and/or calculating the received energy in relation to the emitted energy, a determination may be made as to the content of the gas passing through the device. In one embodiment, the amount of received energy is subtracted from amount of transmitted energy to determine the amount of absorbed energy. The amount of absorbed energy may be correlated to the concentration of a particular gas (such as CO₂) in the exhaled gas. This can then be compared to a known concentration of gas in the air, or as inhaled by the user, using a similar correlation technique to determine CO₂ generated by the user. Similar operation could occur for any molecule or compound in the inhaled/exhaled gas. The analysis is not limited to CO₂.

Continuing to refer to FIG. 2, the example body 203 has an exterior surface 204 having a number of adhesive grips 205 located thereon in order to allow the nasal device 202 to adhere to an interior of the user's nose (e.g., nostrils 140, 142 in FIG. 1B). The body 203 may also have a number of flexible bands 206 extending parallel to the lumen 214 in order to allow the nasal device 202 to be grasped. That is, the bands 206 may flex more than the exterior surface 204 in order to allow the nasal device 202 to be handled and/or more easily inserted into a user's nose.

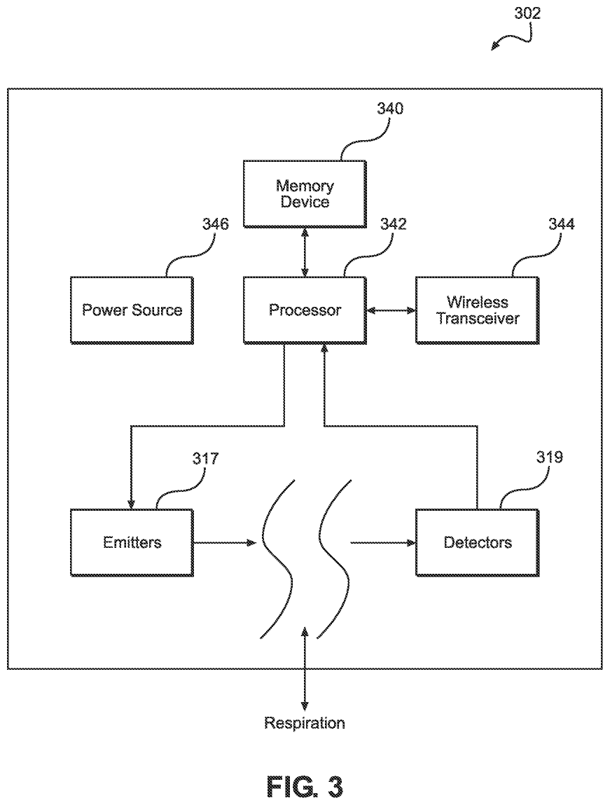

FIG. 3 illustrates one or more internal components of an exemplary nasal device 302, which is structured similar to the nasal devices 102, 202, discussed above, and like numbers represent like features. The nasal device 302 may include a memory device 340 for storing non-transitory machine-readable code, a processor 342, a wireless transceiver 344, and a power source 346 configured to power the processor 342. The components 340, 342, 344, 346 may all be located in compartments of the body of the nasal device 302 in the same manner in which the emitters 117, 127 and detectors 119, 129 are coupled to compartments 113, 115, 123, 125.

The processor 342 may be electrically connected to the transceiver 344, and is configured to execute the machine-readable code stored in the memory device 340. Additionally, the emitters 317 and the detectors 319 may be electrically connected to the processor 342 such that the processor 342 may control the emission and detection of light or any other type of energy discussed above. One or more additional electrical elements, such as drivers, may be located between the processor 342 and the emitters. Information relating to the emitted energy and the detected energy may be gathered and stored in the memory device 340. In addition, the wireless transceiver 344 may be coupled to the body of the nasal device 302 and configured to transmit that information (e.g., data corresponding to at least one of the emitted energy and the detected energy) to external devices (discussed further below). Moreover, the power source 346 may be a battery, respiration generated power source, or any other power source.

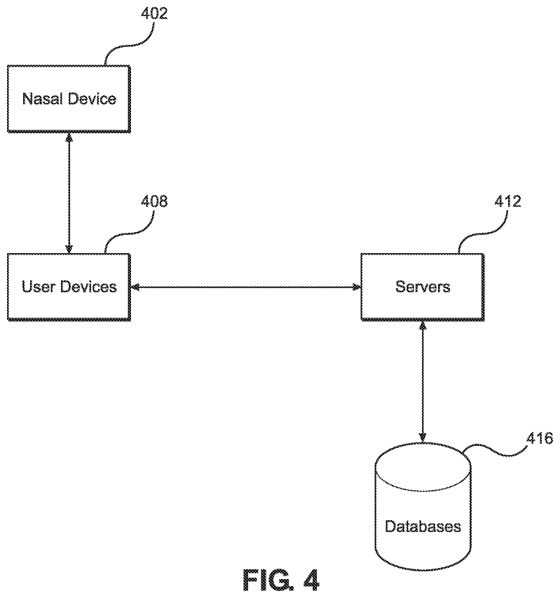

FIG. 4 illustrates an exemplary environment of use of a nasal device 402, structured similar to the nasal devices 102, 202, 302. In FIG. 4, the wireless transceiver of the nasal device 402 may be electronically connected to one or more user devices 408. User devices 408 may include, but are not limited to, a Personal Digital Assistant (“PDA”), cellular telephone, smart phone, tablet PC, wireless electronic pad, an IoT device, a “wearable” electronic device or any other computing device. The electronic communication may be established via Bluetooth® or via network providing internet or signals (e.g., a personal area network, a local area network (“LAN”), a wireless LAN, a wide area network, or any other type of wireless or wired link.). The user devices 408 may, in turn, be electronically connected to one or more servers 412 (or computers) stored in local devices, a cloud, and/or cloud-based servers, as well as other devices 408, which may in turn be connected to their respective databases 416.

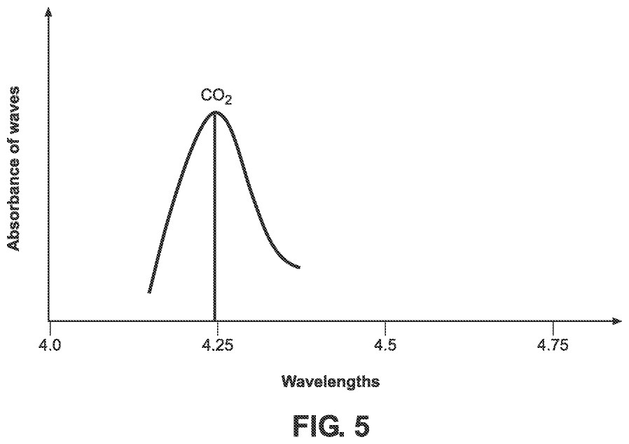

FIG. 5 illustrates an exemplary plot of carbon dioxide absorbance relative to wavelengths. Specifically, LED light having a wavelength between 4.0 micrometers (“μm”) and 4.5 μm may be blocked or absorbed by carbon dioxide molecules, with the highest level of obsorbtion or blockage by carbon dioxide at the wavelength of 4.25 μm. Thus, one embodiment of the emitted energy from the emitters 217 of the nasal device 202 includes light having a wavelength between 4.0 and 4.5 μm. In a preferred embodiment, the wavelength is between 4.20 and 4.30 μm. In a more preferred embodiment, the emitters 217 are configured to emit LED light at 4.25 μm, and a calculation is made to determine carbon dioxide levels based on the fall-off in light intensity across the nasal device gap during exhale. In other embodiments, a wider wavelength spectrum may be sent and the detector configured for a specific wavelength.

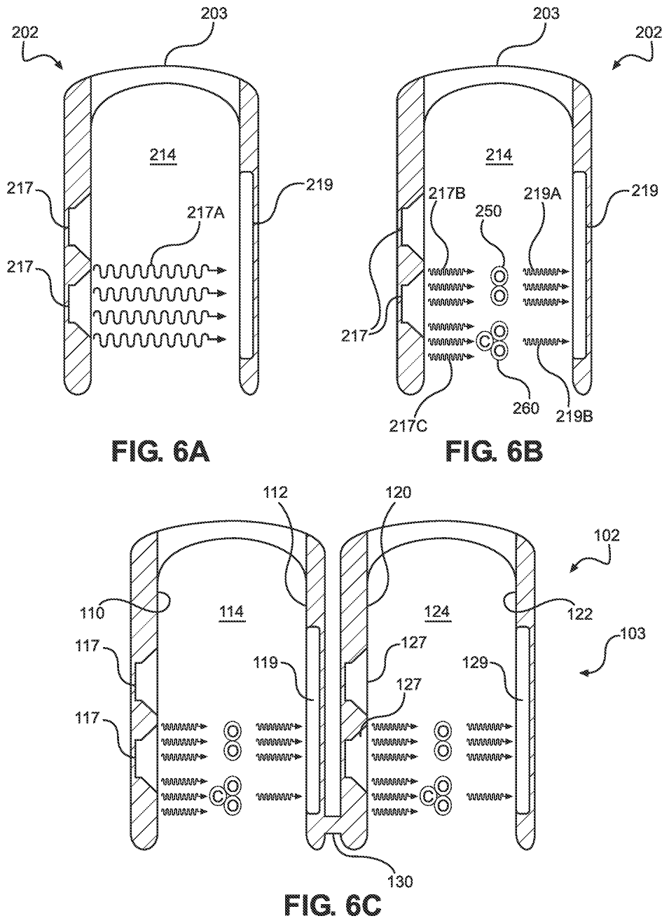

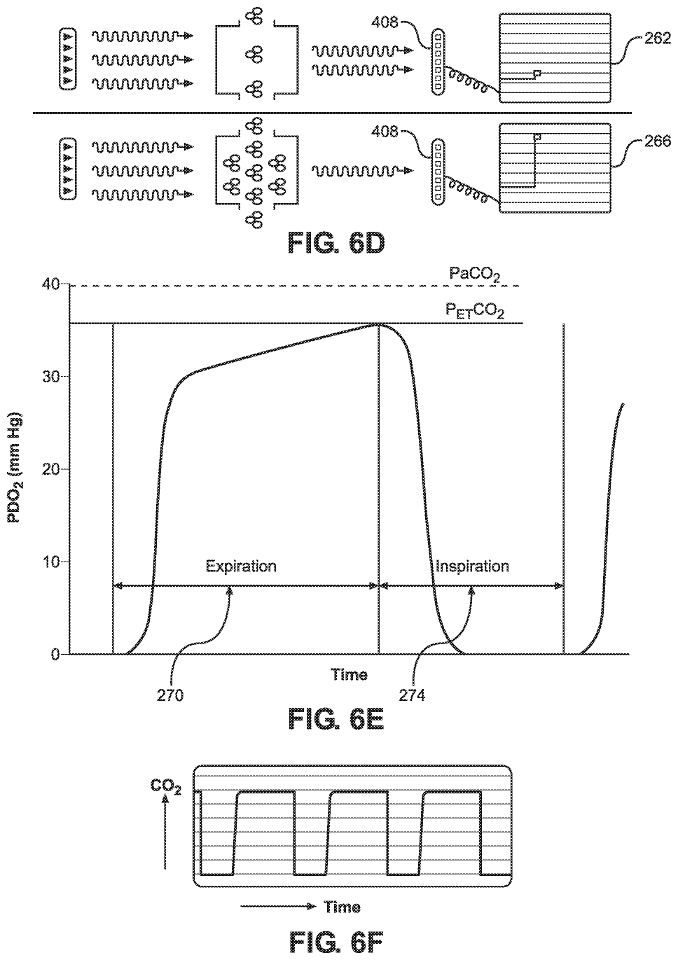

FIG. 6A illustrates an exemplary operation of the nasal device 202 without the presence of gas molecules. As shown in FIG. 6A, the emitters 217 are configured to emit light or any other type of energy 217A, which the detectors 219 are configured to detect and measure. When gas particles, molecules, or atoms are not present in the lumen, there are no molecules to block or absorbe the light or any other type of energy 217A. As such, the detected and emitted energy are the same.

FIG. 6B illustrates an exemplary emission and detection method using wavelength absorption detection when respiration passes through the lumen 214 of the nasal device 202. In FIG. 6B, the nasal device 202 may include one or more emitters 217 on one side and one or more detectors 219 on the other side. The emitters 217 are configured to emit light or any other type of energy 217B, 217C, which the detectors 219 are configured to receive, detect and measure.

As discussed above, in a preferred embodiment, the emitters 217 may be configured to emit LED lights at the wavelength between 4.0 and 4.5 μm, and preferably between 4.20 and 4.30 μm. It is contemplated that energy at other wavelengths may be used based on the gas or molecule of interest. LED light at this wavelength may not be blocked by oxygen 250 traveling through the lumen 214 (such as on inhale through the nose). Thus, the amount of LED light detected 219A will be the same or substantially similar to the emitted light energy 217B. On the other hand, light at the selected wavelength may be blocked by carbon dioxide 260 traveling through the lumen 214 (such as on exhale through the nose). Thus, the amount of LED light detected 219B will be reduced or less than the amount of LED light emitted 217C due to the blockage or absorption by the gas of interest, such as carbon dioxide or the due to the absorption by the molecules of interest. Stated another way, the amount of energy striking the detector will be generally the same during each inhalation because the inhaled air will have the same general concentration of carbon dioxide under normal conditions. However, the exhaled air will have a greater concentration of carbon dioxide and this will be reflected by a reduced amount of energy striking the detector. As the wearer exhales more carbon dioxide (such as due to physical excertion), the amount of enery detected by the detector will be further reduced due to absorption by the greater concentration of carbon dioxide during exhalation. This can be tracked over time to derive the data disclosed herein. It is further contemplated that changes in detected energy during inhalation may reveal changes in gas concentration in the air being inhaled, such as if the inhaled gas has a higher concentration of a gas of interest, which may indicate an unsafe environment.

The wavelength of light may be tuned or selected to correspond to the characteristics of the particular gas being monitored. Carbon dioxide is discussed as an exemplary gas with an associated specific wavelength range, but in other embodiments different gasses of interest may be selected for monitoring and as such other energy wavelengths or types may have to be selected or utilized for the sensor to function as disclosed herein. Accordingly, a first one of the emitters 217 may be configured to emit a first amount of emitted energy and a second one of the emitters 217 may be configured to emit a second amount of emitted energy different than the first amount, thereby allowing the nasal device 202 to measure multiple different gases (e.g., carbon dioxide and oxygen). Emission may be continuous or intermittent.

FIG. 6C illustrates a schematic of the nasal device 102, depicted in FIGS. 1A and 1B. As shown, the device 102 has first and second corresponding emitters 117, 127 and first and second corresponding detectors 119, 129 for each of the interior sides 110, 112, 120, 122 of the first and second lumens 114, 124. Accordingly, it will be appreciated that carbon dioxide and oxygen (other other gas) may be measured in one or more of the nostrils of a user. That is, emitted energy may be emitted from each of the emitters 117, 127 and received at each of the corresponding detectors 119, 129 in order measure various aspects of the respiration.

FIG. 6D illustrates an exemplary graph of the carbon dioxide measured when respiration passes through the lumen 214 of the nasal device 202 in accordance with the disclosed concept. As illustrated in FIG. 4 and discussed above, the nasal device 402 (e.g., and any of the nasal devices 102, 202, 302) may be in communication with one or more user devices 408. The user device 408 may be configured to output a graphical display of the carbon dioxide measurement generated by the nasal device (such as via a software application in communication with a wireless transceiver in the nasal device, which is illustrated as item 344 in FIG. 3 and discussed above).

When no respiration occurs (as illustrated in FIG. 6A), or during inhale (as illustrated by items 217B, 250, 219A in FIG. 6B), the amount of LED light (or any other type of light or energy) detected may be at the same or a substantially similar amount as was emitted. A graphical display of a corresponding low level of carbon dioxide 262 may be generated in response. In contrast, during exhale (as illustrated by items 217C, 260, 219B in FIG. 6B), the amount of LED light (or any other type of light or energy) detected may be significantly less than the amount of LED lights emitted due to the gas on interest, such as carbon dioxide, absorbing of blocking the light from reaching and being detected by the detector. A graphical display of a corresponding higher level of carbon dioxide 266 is generated in response.

FIG. 6E illustrates an exemplary graphical display of the carbon dioxide measured when respiration passes through the lumen 214 of the nasal device 202 during one inhale (inspiration) 274 and one exhale (expiration) 270, where a low level of carbon dioxide is displayed during the inhale, and a high level of carbon dioxide is displayed during the exhale. The horizontal axis represents time while the vertical axis represents carbon dioxide content.

FIG. 6F illustrates an exemplary graphical display of the carbon dioxide measured when respiration passes through the lumen 214 of the nasal device 202 over a period of time, where low levels of carbon dioxide are displayed during inhale, and high levels of carbon dioxide are displayed during exhale. Thus, the innovation disclosed herein can also track respiration rate, inhale rate and duration, exhale rate and duration, periods when inhalation and exhalation is not ocuring and the duration. In addition, the extend of inhalation and exhalation can also be tracked, based on the duration of time that wearer breaths in, which is shown by periods with low CO₂ levels and the duration of time that the CO₂ levels are high, shown by periods of high CO₂ levels.

Accordingly, it will be appreciated that the disclosed concept provides for an improved (e.g., without limitation, able to measure amounts of different gases and thus better protect users) nasal device 102, 202, 302, 402 and method of measuring an aspect of gas flowing through a user's nose (e.g., nostrils 140, 142) using a nasal device 102, 202, 302, 402, in which a number of emitters 117, 127, 217, 317 and detectors 119, 129, 219, 319 are coupled to a body 103, 203 of the nasal device 102, 202, 302, 402. When the emitters 217, 317 emit emitted energy (e.g., without limitation, light having a wavelength between 4.0 and 4.5 micrometers) and the detectors 219, 319 receive detected energy, device electronics can measure amounts of various gases flowing through the lumens by comparing the emitted and detected energies. This is advantageous for a number of reasons.

For example, accurately measuring carbon dioxide levels in users can provide indications of lactic acid levels, thus allowing muscular fatigue and/or weakness to be predicted. It is also contemplated that accurate predictions of carbon dioxide levels with the disclosed nasal devices 102, 202, 302, 402 may also be instrumental in predicting conditions involving other organs and tissues, and/or providing early detection capabilities for specific illnesses and diseases. Additionally, by employing the nasal devices 102, 202, 302, 402 of the disclosed concept, large, fixed/wired capnography apparatuses with nasal canula tubes may be avoided when measuring carbon dioxide levels for patient diagnostics when in hospital beds, in favor of the ambulatory freedom and analysis method afforded by the disclosed nasal devices 102, 202, 302, 402.

In one example embodiment, the method includes the steps of directing the gas through a lumen 214 of the nasal device 202, the nasal device 202 being located in the user's nose; emitting a first amount of energy with an emitter 217 of the nasal device 202, the emitter 217 being coupled to a first interior side 210 of a body 203 of the nasal device 202; detecting a second amount of energy received at a detector 219 of the nasal device 202, the detector 219 being coupled to a second interior side 212 of the body 203 of the nasal device 202, the second interior side 212 being located opposite the first interior side 210; comparing the first amount to the second amount to calculate an absorbed energy amount; generating data based on the absorbed energy amount; and transmitting the data with a transceiver 344 of the nasal device. The method may further include employing the processor 342 to compare the first amount to the second amount, and the generate the data. Furthermore, the method may include receiving the data with a user device 408, and sending the data from the user device 408 through a server 412 to a database 416. Additionally, directing the gas may include directing a flow of oxygen in a first direction through the lumen 214 and directing a flow of carbon dioxide in a second direction through the lumen 214, the second direction being opposite the first direction.

In the above description, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.”

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The present subject matter may be embodied in other specific forms without departing from its spirit or essential characteristics. The described examples are to be considered in all respects only as illustrative and not restrictive. All changes which come within the meaning and range of equivalency of the claims are to be embraced within their scope.

It will be understood that the above described arrangements of apparatus and the method therefrom are merely illustrative of applications of the principles of this invention and many other embodiments and modifications may be made without departing from the spirit and scope of the invention as defined in the claims.

What is claimed is:

1. A nasal device to measure an aspect of gas flowing through on or more of a user's nasal passages, the nasal device comprising:
a body having a first interior side and a second, opposing interior side, the body forming a lumen between the first interior side and the second interior side through which the gas flows into and out of the user's nose;
an emitter coupled to the first interior side and configured to emit emitted energy;
a detector coupled to the second interior side and configured to receive detected energy that was emitted from the emitter; and
a wireless transceiver coupled to the body of the nasal device, the wireless transceiver configured to transmit data corresponding to at least the detected energy.

2. The nasal device according to claim 1, wherein the emitted energy comprises light having a wavelength between 4.0 and 4.5 micrometers.

3. The nasal device according to claim 2, wherein the wavelength is between 4.20 and 4.30 micrometers.

4. The nasal device according to claim 2, further comprising a processor, a memory device, and a power source configured to power the processor, wherein the processor is electrically connected to the transceiver, the emitter, and the detector, and wherein the memory device is electrically connected to the processor and configured to store information related to the emitted energy and the detected energy.

5. The nasal device according to claim 4, wherein the power source is a battery.

6. The nasal device according to claim 4, wherein the gas comprises carbon dioxide, and wherein the emitted energy from the emitter is configured to be blocked by the carbon dioxide.

7. The nasal device according to claim 1, wherein the lumen is a first lumen, wherein the emitter is a first emitter, wherein the detector is a first detector, wherein the body further has a third interior side and a fourth interior side opposite the third interior side, wherein the body further forms a second lumen between the third and fourth interior sides through which the gas flows into and out of the user's nose, wherein the body comprises a bridge connecting the first lumen to the second lumen, wherein the nasal device further comprises a second emitter coupled to the third interior side and configured to emit emitted energy, and a second detector coupled to the fourth interior side and configured to receive detected energy from the second emitter.

8. The nasal device according to claim 1, wherein the emitter is a first emitter, wherein the nasal device further comprises a second emitter coupled to the first interior side and configured to emit an amount of emitted energy different than an amount of the emitted energy emitted from the first emitter.

9. The nasal device according to claim 1, wherein the body further has an exterior surface having a number of adhesive grips disposed thereon in order to allow the nasal device to adhere to an interior of the user's nose.

10. The nasal device according to claim 1, wherein the body further has a number of flexible bands extending parallel to the lumen.

11. A method of measuring an aspect of gas flowing through a user's nose using a nasal device, the method comprising the steps of:
directing the gas through a lumen of the nasal device, the nasal device being disposed in the user's nose;
emitting a first amount of energy with an emitter of the nasal device, the emitter being coupled to a first interior side of a body of the nasal device;
detecting a second amount of energy received at a detector of the nasal device, the detector being coupled to a second interior side of the body of the nasal device, the second interior side being disposed opposite the first interior side;
comparing the first amount to the second amount to calculate an absorbed energy amount;
generating data based on the absorbed energy amount; and
transmitting the data with a transceiver of the nasal device.

12. The method according to claim 11, further comprising employing a processor of the nasal device to compare the first amount to the second amount and to generate data.

13. The method according to claim 12, further comprising receiving the data with a user device.

14. The method according to claim 13, further comprising sending the data from the user device through a server to a database.

15. The method according to claim 11, wherein directing the gas comprises directing a flow of oxygen in a first direction through the lumen and directing a flow of carbon dioxide in a second direction through the lumen, the second direction being opposite the first direction.

16. The method according to claim 11, wherein emitting the first amount of energy comprises emitting light having a wavelength between 4.0 and 4.5 micrometers.

17. The method according to claim 16, wherein emitting the first amount of energy comprises emitting light having a wavelength between 4.20 and 4.30 micrometers.

18. The method according to claim 11, wherein the lumen is a first lumen, wherein the emitter is a first emitter, wherein the detector is a first detector, wherein the body further has a third interior side and a fourth interior side opposite the third interior side, wherein the body further forms a second lumen between the third and fourth interior sides; and wherein the method further comprises:
directing the gas through the second lumen;
providing the body with a bridge connecting the first lumen to the second lumen;
emitting a third amount of energy with a second emitter of the nasal device, the second emitter being coupled to the third interior side;
detecting a fourth amount of energy received at a second detector, the second detector being coupled to the fourth interior side;
comparing the third amount to the fourth amount to calculate a second absorbed energy amount;
generating data based on the second absorbed energy amount; and
transmitting the data based on the second absorbed energy amount with the transceiver.

19. The method according to claim 11, wherein the emitter is a first emitter, wherein the nasal device further comprises a second emitter coupled to the first interior side, and wherein the method further comprises:
emitting a third amount of energy from the second emitter, the third amount being different than the first amount;
detecting a fourth amount of energy received at the detector from the second emitter;
comparing the third amount to the fourth amount to calculate a second absorbed energy amount;
generating data based on the second absorbed energy amount; and
transmitting the data based on the second absorbed energy amount with the transceiver.

20. The method according to claim 11, wherein the body further has an exterior surface having a number of adhesive grips disposed thereon in order to allow the nasal device to adhere to an interior of the user's nose.
